(19) United States
(12) Patent Application Publication
(10) Pub. No.: US 2019/0183470 A1
(43) Pub. Date: Jun. 20, 2019

(54) SURGICAL DEVICE AND METHOD FACILITATING REMOVAL OF TISSUE SPECIMENS

(71) Applicant: COVIDIEN LP, Mansfield, MA (US)

(72) Inventors: ARIFMOHAMAD H. MUJAWAR, SANGLI (IN); SUNNY KUMAR, KORAPUT (IN); MUSHTAQUE SYED AHMED, HYDERABAD (IN)

(21) Appl. No.: 15/842,175

(22) Filed: Dec. 14, 2017

(51) Int. Cl. A61B 17/00; A61B 17/16

(52) U.S. Cl. CPC A61B 17/00234; A61B 17/32002; A61B 17/1611

(57) ABSTRACT

A surgical device includes a housing, a grasper assembly extending distally from the housing, a shaft extending distally from the housing, and a movable handle associated with the housing and operably coupled to the grasper assembly and the shaft. The grasper assembly includes first and second jaw members movable between an open position and a closed position. The shaft is movable relative to the grasper assembly between a retracted position and an extended position. The movable handle is associated with the housing and operably coupled to the grasper assembly and the shaft such that movement of the movable handle relative to the housing from an un-actuated position to an actuated position moves the first and second jaw members from the open position to the closed position, and moves the shaft from the retracted position to the extended position.

### BACKGROUND

### Technical Field

The present disclosure relates to tissue specimen removal and, more particularly, to a surgical device and method facilitating removal of tissue specimens from an internal body cavity.

### Background of Related Art

In minimally-invasive surgical procedures, operations are carried out within an internal body cavity through small entrance openings in the body. The entrance openings may be natural passageways of the body or may be surgically created, for example, by making a small incision into which a cannula is inserted.

Minimally-invasive surgical procedures may be used for partial or total removal of tissue from an internal body cavity. However, the restricted access provided by minimally-invasive openings (natural passageways and/or surgically created openings) presents challenges with respect to removal of large tissue specimens. As such, tissue specimens that are deemed too large for intact removal are manipulated and/or broken down to enable removal from the internal body cavity.

### SUMMARY

As used herein, the term “distal” refers to the portion that is described which is further from a user, while the term “proximal” refers to the portion that is being described which is closer to a user. Further, any or all of the aspects described herein, to the extent consistent, may be used in conjunction with any or all of the other aspects described herein.

Provided in accordance with aspects of the present disclosure is a surgical device including a housing, a grasper assembly, a shaft, and a movable handle. The grasper assembly extends distally from the housing and includes first and second jaw members disposed at a distal end portion thereof. The first and second jaw members are movable relative to one another between an open position and a closed position. The shaft extends distally from the housing and is movable relative to the grasper assembly between a retracted position, wherein the first and second jaw members extend distally from the shaft, and an extended position, wherein the shaft at least partially surrounds the first and second jaw members. The movable handle is associated with the housing and operably coupled to the grasper assembly and the shaft such that movement of the movable handle relative to the housing from an un-actuated position to an actuated position moves the first and second jaw members from the open position to the closed position, and moves the shaft from the retracted position to the extended position.

In an aspect of the present disclosure, the surgical device further includes an electrode associated with the shaft and an activation button mounted on the housing and electrically coupled to the electrode. In such aspects, in the actuated position, the movable handle activates the activation button to supply energy to the electrode.

In another aspect of the present disclosure, the electrode is disposed at an open distal end portion of the shaft. The electrode may be a ring electrode disposed annularly about the open distal end portion of the shaft and/or may be a monopolar electrode.

In still another aspect of the present disclosure, a specimen bag depends from the housing and is disposed in communication with an interior of the shaft.

In yet another aspect of the present disclosure, a plurality of inwardly and proximally-extending barbs is disposed within the shaft.

In still yet another aspect of the present disclosure, a biasing member is configured to bias the movable handle towards the un-actuated position, thereby biasing the shaft towards the retracted position and the grasper assembly towards the open position.

Another surgical device provided in accordance with aspects of the present disclosure includes a housing, a shaft, a grasper assembly, an activation button, and a movable handle. The shaft extends distally from the housing and defines an open distal end portion, a proximal opening, and a lumen extending longitudinally therethrough. The shaft further includes an electrode disposed at the open distal end portion thereof. The grasper assembly includes first and second jaw members configured to grasp tissue therebetween. One of the shaft or the grasper assembly is movable relative to the other and the housing from a retracted position, wherein the shaft is disposed proximally of the first and second jaw members, and an extended position, wherein the shaft at least partially surrounds the first and second jaw members. The activation button is mounted on the housing and electrically coupled to the electrode. The movable handle is associated with the housing and operably coupled to the one of the shaft or the grasper assembly such that movement of the movable handle relative to the housing from an un-actuated position to an actuated position moves the one of the shaft or the grasper assembly from the retracted position to the extended position and activates the activation button to supply energy to the electrode.

In an aspect of the present disclosure, the shaft is movable relative to the housing and the grasper assembly between the retracted and extended positions.

In another aspect of the present disclosure, a first drive assembly is operably coupled between the handle and the shaft such that movement of the movable handle relative to the housing from the un-actuated position to the actuated position moves the shaft from the retracted position to the extended position. Alternatively or additionally, a second drive assembly is operably coupled between the handle and the grasper assembly such that movement of the movable handle relative to the housing from the un-actuated position to the actuated position moves the first and second jaw members relative to one another from an open position to a closed position to grasp tissue therebetween.

In still another aspect of the present disclosure, the electrode is a ring electrode disposed annularly about the open distal end portion of the shaft. Alternatively or additionally, the electrode is a monopolar electrode.

In yet another aspect of the present disclosure, a specimen bag depends from the housing and is disposed in communication with the lumen of the shaft.

In still yet another aspect of the present disclosure, a plurality of inwardly and proximally-extending barbs are disposed within the lumen of the shaft.

In another aspect of the present disclosure, a biasing member is configured to bias the movable handle towards the un-actuated position, thereby biasing the one of the shaft or the grasper assembly towards the retracted position.

A method of surgery provided in accordance with aspects of the present disclosure includes positioning a distal end portion of a surgical device adjacent a tissue specimen and actuating a handle of the surgical device through an actuation stroke. Actuation of the handle through the actuation stroke manipulates a grasper assembly at the distal end portion of the surgical device to grasp a portion of the tissue specimen, energizes a distal electrode of the shaft to at least partially resect the portion of the tissue specimen from a remainder of the tissue specimen, and advances a shaft of the surgical device about the grasper assembly to at least partially receive the resected portion of the tissue specimen within the shaft.

In an aspect of the present disclosure, the method further includes moving the resected portion of the tissue specimen proximally through the shaft and into a specimen bag operably coupled to the shaft.

In an aspect of the present disclosure, the method further includes engaging the resected portion of the tissue specimen with at least one barb disposed within the shaft to inhibit distal movement of the resected portion of the tissue specimen relative to the shaft.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements and:

FIG. 1 is a side view of a surgical device provided in accordance with the present disclosure, configured to facilitate removal of tissue specimens from an internal body cavity;

FIG. 2 is a longitudinal, cross-sectional view of the surgical device of FIG. 1;

FIG. 3A is a longitudinal, cross-sectional view of a distal end portion of the surgical device of FIG. 1 positioned adjacent a tissue specimen to be removed;

FIG. 3B is a longitudinal, cross-sectional view of the distal end portion of the surgical device of FIG. 1 grasping and drawing a portion of the tissue specimen into the interior thereof; and

FIG. 4 is a longitudinal, cross-sectional view of an intermediate portion of the surgical device of FIG. 1 illustrating portions of tissue moving proximally through the surgical device and into a specimen bag thereof.

### DETAILED DESCRIPTION

The present disclosure provides a surgical device and method facilitating removal of tissue specimens from an internal body cavity.

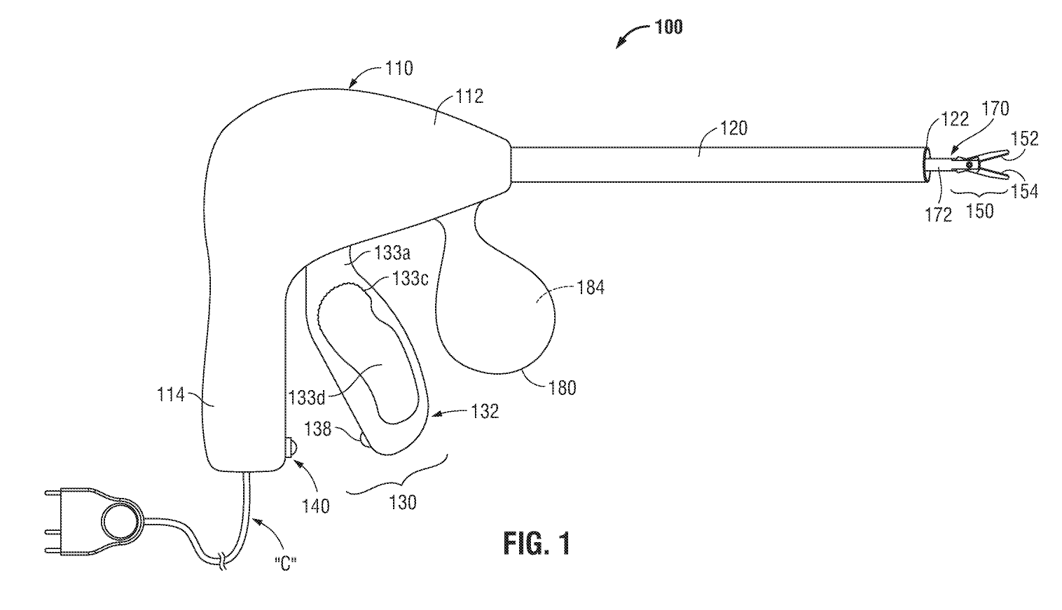

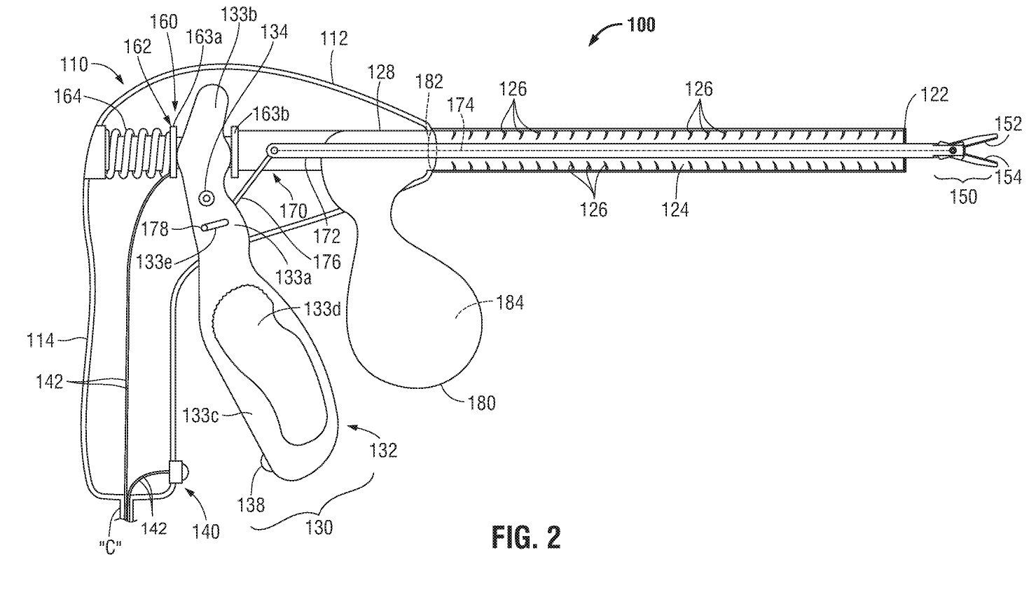

Turning to FIGS. 1 and 2, a surgical device 100 provided in accordance with the present disclosure is shown generally including a housing 110, a shaft 120 extending distally from housing 110, a handle assembly 130 operably coupled to housing 110, an activation button 140 operably coupled to housing 110, a grasper assembly 150 distally-spaced from housing 110, a first drive assembly 160 disposed within housing 110 and operably coupling handle assembly 130 with shaft 120, a second drive assembly 170 extending through housing 110 and shaft 120 and operably coupling grasper assembly 150 with handle assembly 130, and a specimen bag 180 operably supported by housing 110. Surgical device 100 further includes an electrosurgical cable “C” configured to connect surgical device 100 to a source of electrosurgical energy (not shown) to enable selectively delivery of energy to distal electrode 122 of shaft 120, e.g., upon activation of activation button 140, as detailed below.

Housing 110 includes a body portion 112 and a fixed handle 114 that depends from body portion 112 to define the fixed handle 114 of handle assembly 130. Housing 110 houses the internal operating components of surgical device 100, as detailed below.

Shaft 120 extends distally from housing 110 and is movably coupled thereto, as detailed below. Shaft 120, as noted above, includes a distal electrode 122. Distal electrode 122 is configured as a ring electrode disposed about the annular perimeter of shaft 120 at the distal end portion thereof, although other configurations are also contemplated. Distal electrode 122 may be configured to receive monopolar energy and serve as an active electrode for use with a remote return pad (not shown). Alternatively, distal electrode 122 may define one electrode in a bipolar configuration with an electrically-isolated component(s) of surgical device 100, e.g., another portion of shaft 120, one or both of jaw members 152, 154 of grasper assembly 150, etc. Distal electrode 122 is coupled to the source of electrosurgical energy (not shown) and activation button 140 by way of lead wires 142.

Shaft 120 further defines a longitudinally-extending lumen 124 therethrough and includes a plurality of spaced-apart barbs 126 extending from an interior surface of shaft 120 inwardly into lumen 124. Barbs 126 may be arranged in longitudinally-extending rows spaced-apart about the annular interior of shaft 120, or may be arranged in any other suitable manner. Further, barbs 126 may extend over only a portion or over the entire length of shaft 120. Barbs 126 are angled proximally such that tissue may more readily pass proximally over barbs 126 but is inhibited from passing distally over barbs 126.

Shaft 120 further defines a proximal opening 128 disposed towards the proximal end portion thereof. Proximal opening 128 may be defined longitudinally through an open proximal end of shaft 120, or may be defined transversely through shaft 120 towards the proximal end thereof (as shown). Proximal opening 128 is configured to receive or otherwise communicate with an open end 182 of specimen bag 180. More specifically, open end 182 of specimen bag 180 extends through proximal opening 128 of shaft 120 into lumen 124 of shaft 120 and is sealed therein such that tissue passing proximally through lumen 124 of shaft 120 is routed through open end 182 of specimen bag 180 and into interior 184 of specimen bag 180. To this end, specimen bag 180 depends from housing 110 such that gravity facilitates passage of tissue from lumen 124 of shaft 120 into interior 184 of specimen bag 180.

Continuing with reference to FIGS. 1 and 2, handle assembly 130 includes fixed handle 114 and a movable handle 132. Movable handle 132, more specifically, includes an intermediate portion 133a pivotably coupled to housing 110 within housing 110 via a pivot pin 134. Movable handle 132 further includes a bifurcated flange portion 133b extending from intermediate portion 133a further into housing 110 and a grasping portion 133c extending from intermediate portion 133a in an opposite direction as compared to bifurcated flange portion 133b. Grasping portion 133c extends externally from housing 110 and defines a finger loop 133d to facilitate grasping thereof. Intermediate portion 133a of movable handle 132 further defines a cam slot 133e therethrough on the grasping-portion side of pivot pin 134.

Bifurcated flange portion 133b of movable handle 132 surrounds and is captured between annular rims 163a, 163b of mandrel 162 of first drive assembly 160. Mandrel 162, in turn, is secured to the proximal end portion of shaft 120 such that translation of mandrel 162 through housing 110 likewise translates shaft 120 relative to housing 110. First drive assembly 160 further includes a biasing member 164 configured to bias mandrel 162 proximally, thereby biasing shaft 120 proximally and movable handle 132 towards an un-actuated position, wherein movable handle 132 is spaced-apart from fixed handle 114. As an alternative to first drive assembly 160 coupling movable handle 132 with shaft 120 to enable movement of shaft 120 relative to housing 110 (and, thus, grasper assembly 150), first drive assembly 160 may couple movable handle 132 with outer shaft 174 of second drive assembly 170, similarly as above, to enable movement of outer shaft 174 and, thus, grasper assembly 150 relative to housing 110 (and, thus, shaft 120).

Movable handle 132 is further operably coupled to grasper assembly 150 via cam slot 133e and second drive assembly 170. Second drive assembly 170 includes an outer shaft 172 fixed relative to housing 110 and supporting grasper assembly 150 at a distal end portion thereof, and inner drive shaft 174 slidably disposed within outer shaft 174 and operably coupled to jaw members 152, 154 of grasper assembly 150 at a distal end portion thereof, and a linkage 176 operably coupled between cam slot 133e and a proximal end portion of inner drive shaft 174. More specifically, a cam pin 178 extending transversely from the proximal end portion of linkage 176 is received within cam slot 133e such that, upon pivoting of movable handle 132 about pivot pin 134 and relative to fixed handle 114 from the un-actuated position towards the actuated position, linkage 176 is pulled proximally, thereby pulling inner drive shaft 174 proximally through and relative to outer shaft 172. Inner drive shaft 174 is operably coupled to jaw members 152, 154 of grasper assembly 150, e.g., via a cam-slot mechanism (not shown), such that proximal movement of inner drive shaft 174 through outer shaft 172 pivots jaw members 152, 154 relative to one another from an open positon towards a closed position to grasp tissue therebetween.

Movable handle 132 additionally includes a protrusion 138 extending proximally from grasping portion 133c thereof. Protrusion 138 is configured to align with activation button 140 to enable activation of activation button 140 via urging from protrusion 138 upon pivoting of movable handle 132 about pivot pin 134 and relative to fixed handle 114 from the un-actuated position to the actuated position, wherein movable handle 132 is approximated relative fixed handle 134. Activation button 140, in turn, is electrically coupled to the source of electrosurgical energy (not shown) and distal electrode 122 via lead wires 142 to enable selectively delivery of energy from the source of electrosurgical energy (not shown) to distal electrode 122 upon activation of activation button 140.

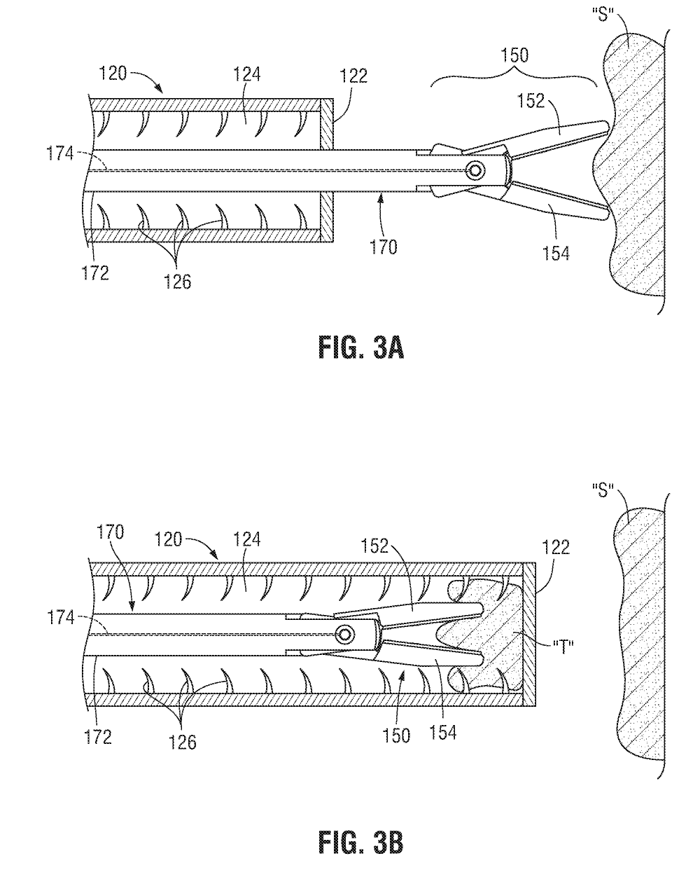

Referring still to FIGS. 1 and 2, as a result of the above-detailed configuration, pivoting of movable handle 132 about pivot pin 134 and relative to fixed handle 114 from the un-actuated position to the actuated position: urges bifurcated flange portion 133b of movable handle 132 distally, thereby urging mandrel 162 and shaft 120 distally relative to housing 110 such that shaft 120 is moved from a retracted position (FIG. 3A), wherein grasper assembly 150 extends distally from shaft 120, to an extended position (FIG. 3B), wherein shaft 120 is disposed at least partially about grasper assembly 150; pulls linkage 176 proximally, thereby pulling inner drive shaft 174 proximally to pivot jaw members 152, 154 relative to one another from an open positon towards a closed position to grasp tissue therebetween; and urges protrusion 138 into activation button 140 to activate activation button 140, thereby initiating the supply of energy to distal electrode 122.

Referring to FIGS. 1-4, the use and operation of surgical device 100 is described. Initially, movable handle 132 is disposed in the un-actuated position under the bias of biasing member 164 and, as such, shaft 120 is disposed in the retracted position (FIG. 3A), wherein grasper assembly 150 extends distally from shaft 120. Further, in the un-actuated position of movable handle 132, jaw members 152, 154 of grasper assembly 150 are disposed in the open position (FIG. 3A) and distal electrode 122 is un-energized.

With reference to FIG. 3A, in conjunction with FIG. 2, with movable handle 132 disposed in the un-actuated position, surgical device 100 is manipulated into position adjacent a tissue specimen “S” to be removed such that jaw members 152, 154 of grasper assembly 150 are positioned adjacent the tissue specimen “S.”

With additional reference to FIG. 3B, once jaw members 152, 154 of grasper assembly 150 are positioned adjacent the tissue specimen “S” as detailed above, movable handle 132 may be moved from the un-actuated position to the actuated position. Movement of movable handle 132 through an actuation stroke from the un-actuated position to the actuated position pivots jaw members 152, 154 of grasper assembly 150 relative to one another from the open positon towards the closed position to grasp the tissue specimen “S,” and also advances shaft 120 from the retracted position to the extended position. As such, shaft 120 is extended to at least partially surround grasper assembly 150 and the portion of the tissue specimen “S” grasped thereby. In addition, movement of movable handle 132 through the actuation stroke urges protrusion 138 into activation button 140 to activate activation button 140, thereby initiating the supply of energy to distal electrode 122 such that, as shaft 120 is advanced about the portion of the tissue specimen “S” grasped by jaw members 152, 154 of grasper assembly 150, the portion of tissue is electromechanically resected, partially or wholly, from the remainder of the tissue specimen “S.” More specifically, the portion of tissue may be resected fully from the tissue specimen “S” to form a tissue segment “T” (as shown), or may be only partially resected such that the portion of the tissue specimen “S” defines a tissue strip capable of passing through lumen 124 of shaft 120.

Once the tissue segment “T” is resected and received within lumen 124 of shaft 120, movable handle 132 may be returned towards the un-actuated position or released, allowing movable handle 132 to be returned under bias of biasing member 164. Return of movable handle to the un-actuated position pivots jaw members 152, 154 of grasper assembly 150 relative to one another from the closed positon back towards the open position to release the grasped tissue segment “T,” and also retracts shaft 120 from the extended position back towards the retracted position. Although shaft 120 is retracted proximally, the tissue segment “T” is maintained within shaft 120 via engagement of the tissue segment “T” with barbs 126. Thus, once the tissue segment “T” is disposed within shaft 120, barbs 126 permit the tissue segment “T” to move proximally through shaft 120 but inhibit the tissue segment “T” from moving distally therethrough.

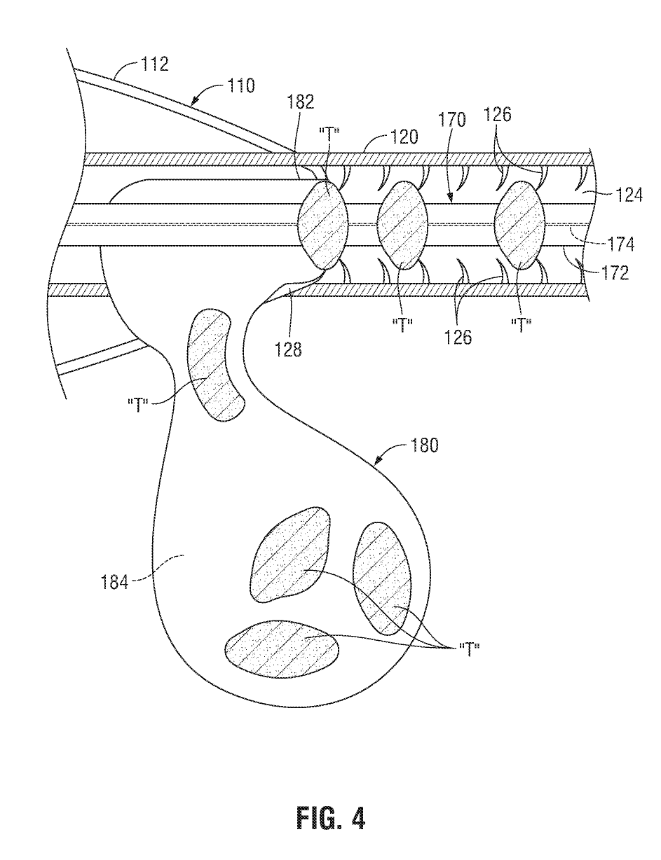

Referring also to FIG. 4, with moveable handle 132 returned to the un-actuated position, the above-detailed use may be repeated such that movable handle 132 is actuated through one or more additional actuation strokes to grasp, resect, and draw additional tissue segments “T” into lumen 124 of shaft 120. As more tissue segments “T” are drawn into lumen 124 of shaft 120 (or, as more length of the tissue strip is drawn into lumen 124 of shaft 120, in embodiments where tissue is not fully resected), the more-distal tissue segments “T” urge the more-proximal tissue segments “T” further proximally through lumen 124 of shaft 120. Ultimately, as each tissue segment “T” reaches proximal opening 128 of shaft 120, the tissue segment “T” falls, under gravity, through open end 182 of specimen bag 180 and into interior 184 thereof, thus collecting the tissue segments “T” for testing and/or safe disposal.

Referring generally to FIGS. 1-4, as an alternative to manual actuation via movable handle 132, the above-detailed surgical device 100 may alternatively be configured for use with a robotic surgical system configured, in response to receipt of an actuation signal, to actuate first and second drive assemblies 160, 170 and activation button 140 such that, similarly as detailed above, each actuation pivots jaw members 152, 154 of grasper assembly 150 relative to one another from the open positon towards the closed position to grasp a tissue specimen “S,” advances shaft 120 distally to at least partially surround grasper assembly 150 and the portion of the tissue specimen “S” grasped thereby, and activates activation button 140 to supply energy to distal electrode 122 to resect the portion of the tissue specimen “S” grasped by jaw members 152, 154. In addition, as also detailed above, a specimen bag 180 may be provided to collect the tissue segments “T” for testing and/or safe disposal.

The robotic surgical system may employ various robotic elements to assist the surgeon and allow remote operation (or partial remote operation). More specifically, various robotic arms, gears, cams, pulleys, electric and mechanical motors, etc. may be employed for this purpose and may be designed with the robotic surgical system to assist the surgeon during the course of an operation or treatment. The robotic surgical system may include remotely steerable systems, automatically flexible surgical systems, remotely flexible surgical systems, remotely articulating surgical systems, wireless surgical systems, modular or selectively configurable remotely operated surgical systems, etc.

The robotic surgical system may be employed with one or more consoles that are next to the operating theater or located in a remote location. In this instance, one team of surgeons or nurses may prep the patient for surgery and configure the robotic surgical system with the surgical device disclosed herein while another surgeon (or group of surgeons) remotely control the surgical device via the robotic surgical system. As can be appreciated, a highly skilled surgeon may perform multiple operations in multiple locations without leaving his/her remote console which can be both economically advantageous and a benefit to the patient or a series of patients.

The robotic arms of the robotic surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various drawings, those skilled in the art will appreciate that certain modifications can be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical device, comprising:
a housing;
a grasper assembly extending distally from the housing, the grasper assembly including first and second jaw members disposed at a distal end portion thereof, the first and second jaw members movable relative to one another between an open position and a closed position;
a shaft extending distally from the housing and movable relative to the grasper assembly between a retracted position, wherein the first and second jaw members extend distally from the shaft, and an extended position, wherein the shaft at least partially surrounds the first and second jaw members; and
a movable handle associated with the housing and operably coupled to the grasper assembly and the shaft such that movement of the movable handle relative to the housing from an un-actuated position to an actuated position moves the first and second jaw members from the open position to the closed position, and moves the shaft from the retracted position to the extended position.

2. The surgical device according to claim 1, further comprising an electrode associated with the shaft and an activation button mounted on the housing and electrically coupled to the electrode, wherein, in the actuated position, the movable handle activates the activation button to supply energy to the electrode.

3. The surgical device according to claim 2, wherein the electrode is disposed at an open distal end portion of the shaft.

4. The surgical device according to claim 3, wherein the electrode is a ring electrode disposed annularly about the open distal end portion of the shaft.

5. The surgical device according to claim 2, wherein the electrode is a monopolar electrode.

6. The surgical device according to claim 1, further comprising a specimen bag depending from the housing, the specimen bag disposed in communication with an interior of the shaft.

7. The surgical device according to claim 1, further comprising a plurality of inwardly and proximally-extending barbs disposed within the shaft.

8. The surgical device according to claim 1, further comprising a biasing member configured to bias the movable handle towards the un-actuated position, thereby biasing the shaft towards the retracted position and the grasper assembly towards the open position.

9. A surgical device, comprising:
a housing;
a shaft extending distally from the housing, the shaft defining an open distal end portion, a proximal opening, and a lumen extending longitudinally therethrough, the shaft including electrode disposed at the open distal end portion of the shaft;
a grasper assembly including first and second jaw members configured to grasp tissue therebetween, wherein one of the shaft or the grasper assembly is movable relative to the other and the housing from a retracted position, wherein the shaft is disposed proximally of the first and second jaw members, and an extended position, wherein the shaft at least partially surrounds the first and second jaw members;
an activation button mounted on the housing and electrically coupled to the electrode; and
a movable handle associated with the housing and operably coupled to the one of the shaft or the grasper assembly such that movement of the movable handle relative to the housing from an un-actuated position to an actuated position moves the one of the shaft or the grasper assembly from the retracted position to the extended position and activates the activation button to supply energy to the electrode.

10. The surgical device according to claim 9, wherein the shaft is movable relative to the housing and the grasper assembly between the retracted and extended positions.

11. The surgical device according to claim 10 further comprising a first drive assembly operably coupled between the handle and the shaft such that movement of the movable handle relative to the housing from the un-actuated position to the actuated position moves the shaft from the retracted position to the extended position.

12. The surgical device according to claim 9, further comprising a second drive assembly operably coupled between the handle and the grasper assembly such that movement of the movable handle relative to the housing from the un-actuated position to the actuated position moves the first and second jaw members relative to one another from an open position to a closed position to grasp tissue therebetween.

13. The surgical device according to claim 9, the electrode is a ring electrode disposed annularly about the open distal end portion of the shaft.

14. The surgical device according to claim 9, wherein the electrode is a monopolar electrode.

15. The surgical device according to claim 9, further comprising a specimen bag depending from the housing, the specimen bag disposed in communication with the lumen of the shaft.

16. The surgical device according to claim 9, further comprising a plurality of inwardly and proximally-extending barbs disposed within the lumen of the shaft.

17. The surgical device according to claim 9, further comprising a biasing member configured to bias the movable handle towards the un-actuated position, thereby biasing the one of the shaft or the grasper assembly towards the retracted position.

18. A method of surgery, comprising:
positioning a distal end portion of a surgical device adjacent a tissue specimen; and
actuating a handle of the surgical device through an actuation stroke, wherein actuation of the handle through the actuation stroke:
manipulates a grasper assembly at the distal end portion of the surgical device to grasp a portion of the tissue specimen;
energizes a distal electrode of the shaft to at least partially resect the portion of the tissue specimen from a remainder of the tissue specimen; and
advances a shaft of the surgical device about the grasper assembly to at least partially receive the resected portion of the tissue specimen within the shaft.

19. The method according to claim 18, further comprising moving the resected portion of the tissue specimen proximally through the shaft and into a specimen bag operably coupled to the shaft.

20. The method according to claim 18, further comprising engaging the resected portion of the tissue specimen with at least one barb disposed within the shaft to inhibit distal movement of the resected portion of the tissue specimen relative to the shaft.
